(19) United States
(12) Patent Application Publication
(10) Pub. No.: US 2016/0216272 A1
(43) Pub. Date: Jul. 28, 2016

(54) KIT AND METHOD FOR THE LABELLING OF BIOMOLECULES

(71) Applicant: Cyanagen S.r.l., Bologna (IT)

(72) Inventor: Leopoldo DELLA CIANA, Bologna (IT)

(21) Appl. No.: 15/086,043

(22) Filed: Mar. 30, 2016

Related U.S. Application Data
(62) Division of application No. 12/908,145, filed on Oct. 20, 2010.

(30) Foreign Application Priority Data
Oct. 21, 2009 (IT) BO2009A000682

(51) Int. Cl. G01N 33/58; C07H 1/00; C12Q 1/68; C07K 1/13

(52) U.S. Cl. CPC G01N 33/582; C07K 1/13; C07H 1/00; C12Q 1/6806

(57) ABSTRACT

The present invention relates to a kit for the labelling of biomolecules bearing reactive amino or hydroxyl groups. The kit consists of a Reagent A and a Reagent B, individually packaged, comprising: a mixture of a carboxylated labelling compound and a tertiary amine (Reagent A); and a coupling reagent (Reagent B). Upon contacting Reagent A with Reagent B, the carboxylated labelling compound is activated in-situ by the coupling reagent (a guanidinium, or uranium salt) in the presence of the tertiary amine. The active form of the carboxylated labelling compound is reacted with a biomolecules bearing reactive amino or hydroxyl groups, with formation of a stable covalent bond between the carboxylated labelling compound and the biomolecule.

### FIELD OF THE INVENTION

The present disclosure relates to a kit and a method for the labelling of biomolecules.

### STATE OF THE ART

The labelling of biomolecules bearing amino groups is of great importance in many areas of biology and biotechnology, in particular in the fields of diagnostics, immunoassays, nucleic acid assays, bioimaging, and therapeutics.

For these purposes, the labelling compound is often an isothiocyanate, a sulfonyl chloride, or an active ester. Active esters are currently preferred, in particular N-hydrosuccinimide (NHS) esters or sulfo N-hydroxysuccinimide (sulfo-NHS) esters, see, e.g., Hermanson, “Bioconjugated Techniques”, Academic Press (1996). This type of active esters is prepared from the corresponding carboxylic acid and N-hydroxysuccimides by means of carbodiimides, such as dicylohexylcarbodiimide (DDC) or N-(3-dimethylaminopropyl) N′-ethylcarbodiimide (EDC), or carbonylimidazole. Alternatively, the carboxylic acid can be converted into the corresponding N-hydroxysuccinimide (NHS) or sulfo-NHS ester by means of disuccinimidylcarbonate. The active ester is isolated, generally as a solid, and stored under dry conditions.

In most cases, NHS and sulfa-NHS esters are very moisture sensitive and cannot be easily purified by crystallization or chromatography. Therefore, they often contain considerable and variable amounts of impurities, especially of the starting carboxylic acid. In some instances, isolation of the active ester produces a large increase in non-specific binding upon conjugation e.g., the NHS ester of the chemiluminescent label 7-(4-aminobutyl-N-ethyl)naphthalene-12-dicarboxylic acid hydrazide hemisuccinic acid(ABEN-H) Chapter 3, p. 146, in “Luminescence Immunoassays and Molecular Applications”, Van Dyke and Van Dyke Eds. CRC Press (1990).

NHS and sulfo-NHS active esters of certain compounds, especially fluorescent dyes, such as cyanines, must be stored under very dry conditions. However, unlike their carboxylic acid precursors, such active esters are often sensitive to electrostatic charges, and therefore difficult to weigh. Aliquoting of such active esters, which are often very expensive to fabricate, is therefore cumbersome; in addition even their DMF or DMSO solutions are of limited stability, being sensitive to even traces of moisture.

From the above discussion, it appears that the current use of isolated active esters to label biomolecules is often unreliable and wasteful. Because both the active ester and the biomolecules to be labelled are usually very costly, these problems represent a serious economic loss.

Therefore, it would be desirable to have a method for the rapid, quantitative in situ activation of a moisture stable, purifiable carboxylated labelling precursor compound.

### SUMMARY OF THE INVENTION

Taking into consideration these premises, it is therefore felt the need for ameliorative solutions, more effective for labelling biomolecules bearing reactive amino and hydroxyl groups.

According to the invention, the above object is achieved thanks to the solution as called for in the claims that follow, which are an integral part of the disclosure of the invention as provided herein.

An embodiment of the present invention concerns a kit for labelling biomolecules bearing reactive amino and hydroxyl groups comprising a first component (Reagent A) and a second component (Reagent B), wherein said first component comprises a mixture of a carboxylated labelling compound and a tertiary amine, and wherein said second component comprises:

i) a guanidinium salt of formula (a)

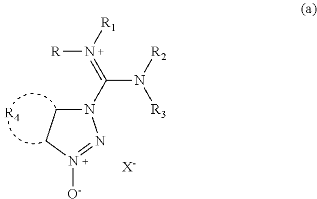

wherein

R, R₁, R₂, R₃ are independently selected from hydrogen, substituted or unsubstituted alkyl having 1 to 6 carbon atoms, substituted or unsubstituted alkenyl having 2 to 6 carbon atoms and substituted or unsubstituted alkynyl having 2 to 6 carbon atoms, or

R and R₁, when taken together, or R₂ and R₃, when taken together, constitute a substituted or unsubstituted 5- to 7-membered heterocyclic ring including the nitrogen atom to which they are attached, or R and R₂, when taken together, constitute a substituted or unsubstituted 5- to 7-membered heterocyclic ring including the guanidinium group to which they are attached, and

R₄ represents a substituted or unsubstituted 6- to 10-membered aromatic or heteroaromatic ring, and

X⁻ is an anion;

or

ii) an uranium salt of formula (b)

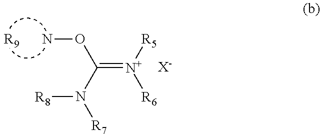

wherein

R₅, R₆, R₇, R₈ are independently selected from hydrogen, substituted or unsubstituted alkyl having 1 to 6 carbon atoms, substituted or unsubstituted alkenyl having 2 to 6 carbon atoms and substituted or unsubstituted alkynyl having 2 to 6 carbon atoms, or

R₅and R₆, when taken together, or R₇ and R₈, when taken together, constitute a substituted or unsubstituted 5- to 7-membered heterocyclic ring including the nitrogen atom to which they are attached, or R₆ and R₇ when taken together, constitute a substituted or unsubstituted 5- to 7-membered heterocyclic ring including the uronium group to which they are attached,

R₉ represents a substituted or unsubstituted 5- to 7-membered heterocyclic ring including the nitrogen atom to which is attached, and

X⁻ is an anion.

A further embodiment of the present invention concerns use of a mixture of a carboxylated labelling compound and a tertiary amine (Reagent A), and a guanidinium salt of formula (a) or a uronium salt of formula (b) (as defined above) (Reagent B) for labelling biomolecules bearing reactive amino or hydroxyl groups.

A still further embodiment of the present invention concerns a method for labelling biomolecules bearing reactive amino or hydroxyl groups with a carboxylated labelling compound, comprising i) providing a mixture of the carboxylated labelling compound and a tertiary amine (Reagent A), and one between a guanidinium salt of formula (a) or a uronium salt of formula (b) (as defined above) (Reagent B), ii) contacting the mixture and the guanidinium salt or the uronium salt obtaining an activated carboxylated labelling compound; iii) contacting the activated carboxylated labelling compound with the biomolecule obtaining a biomolecule labelled with the carboxylated labelling compound.

Both Reagent A and Reagent B are chemically stable for a very long period of time, when stored in the absence of moisture.

Reagent A can be a solution in a suitable solvent, such as a water miscible, polar aprotic solvent such as DMF, DMSO, and the like. However, it can be, in most cases evaporated to a solid.

Reagent B consists of a guanidinium or uronium coupling reagent (activator), such as N,N,N′,N′-tetramethyl-O—(N-succinimidyl)uranium tetrafluoroborate (TSTU), These compounds are powerful activators well known in peptide synthesis, e.g., Knorr et al, Tet. Lett. 1990, 30, 1927; Bannwarth and Knorr, Tet. Lett. 1991, 32, 1157; Carpino et al. J. Org. Chem., 1994, 59, 695-698; U.S. Pat. No. 6,825,347) either solid, or dissolved in a suitable solvent such as a water miscible, polar aprotic solvent such as DMF, DMSO, and the like.

At least one of the two reagents must be in solution form.

Upon contacting Reagent A and Reagent B, an activated form of the carboxylated labelling compound is rapidly formed, in general, quantitatively. This activated form can then be used to reliably and reproducibly label a biomolecule bearing reactive amino or hydroxyl groups. Reaction byproducts are easily removed by chromatography, dialysis, or ultrafiltration techniques.

### BRIEF DESCRIPTION OF THE FIGURES

FIG. 1 illustrates the general formulae of the uronium (a) and guanidinium (b) salts used as activators according to the present invention.

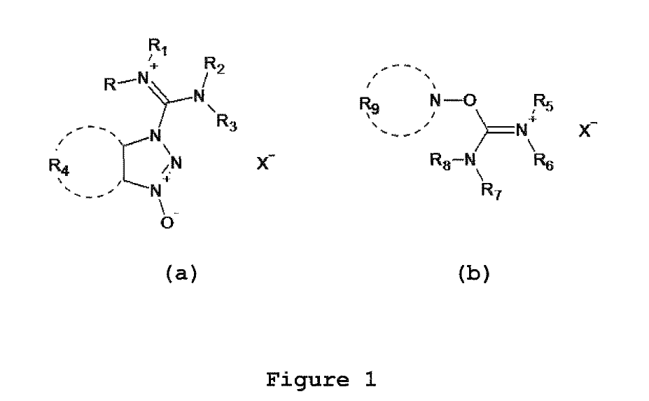

FIG. 2 graphically illustrates the steps required for the use of the antibody labelling kits prepared according to the present invention, namely (a) Activated Dye Solution Preparation; (b) Antibody Solution Preparation; (c) Conjugation Procedure; (d) Purification and Isolation of the Conjugate.

### DETAILED DESCRIPTION OF THE INVENTION

The kits prepared according to the method of the present invention can be used in many biological and biotechnological procedures. For example, they can be used to label antibodies or other proteins. The labelled antibodies or proteins can be used as reagents in immunoassays or other diagnostic techniques such as flow cytometry, or in bioimaging, or as therapeutic reagents. The labelling kits can be successfully employed in proteomics techniques, such as differential gel electrophoresis.

Similarly, the kits of this invention can be used to label amino-modified nucleotides to be used in nucleic acid assays.

The kits prepared according to this invention overcome the difficulties of the presently used active ester, by providing the labelling compound, containing a carboxyl group, and the coupling reagent in separate packages, which can be stored for a prolonged period, without degradation. No weighing of costly, moisture sensitive material is required, nor aliquoting of rapidly decomposing solutions.

In more detail, the kit consists of a Reagent A and a Reagent B, individually packaged, comprising; a mixture of a carboxylated labelling compound and a tertiary amine (Reagent A); and a coupling reagent (Reagent B). The two reagents are carefully formulated and proportioned for a given amount of biomolecules to be labeled.

Reagent A consists of a mixture of a carboxylated compound and a tertiary amine, solid or in solution.

The carboxylated compound can be a luminescent compound containing a carboxylic moiety and said luminescent compound belongs to the class of coumarines, fluoresceines and rhodamines, polymethines including cyanines and merocyanines, oxazines, thiazines, phthalo-and naphthalocyanines, diazaindacenes, transition metal complexes and chelates, such as luminescent complexes of Ru (II), Os (II) and Ir (III) with aromatic diazines, or luminescent complexes of Eu (III) and Tb (III).

The carboxylated labelling compound can be a luminogenic compound containing a carboxylic moiety and said luminescent compound can be a carboxyl functionalized derivative of luminol and it analogues, e.g. hemisuccinamides of 5-amino-2,3-dihydrophthalazine-1,4-dione (or aminobutilisoluminol, ABEI), 6-amino-2,3-dihydrophthalazine-1,4-dione, 9-amino-2,3-dihydrobenzo[f]phthalazine-1,4-dione (or aminobutyl naphthol, ABEN), or other phthalazine derivatives, or carboxyl functionalized acridinium esters.

The tertiary amine of Reagent A can be triethylamine, N,N-diisopropylethylamine, pyridine, N,N-dimethylamino pyridine (DMAP), 4-piperidylpyridine (PPY), 2,4,6-trimethylpyridine(collidine), 4-methylmorpholine, 4-ethylmorpholine, 4-morpholinopyridine.

Reagent B is a coupling reagent comprising a guanidinium (a) or uronium (b) salt.

The guanidinium salt presents chemical formula (a):

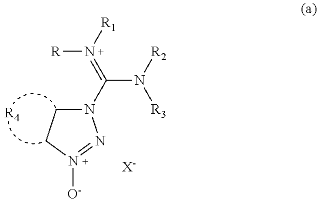

wherein

R, R₁, R₂, R₃ are independently selected from hydrogen, substituted or unsubstituted alkyl having 1 to 6 carbon atoms, substituted or unsubstituted alkenyl having 2 to 6 carbon atoms and substituted or unsubstituted alkenyl having 2 to 6 carbon atoms, or

R and R₁, when taken together, or R₂ and R₃, when taken together, constitute a substituted or unsubstituted 5- to 7-membered heterocyclic ring including the nitrogen atom to which they are attached, or R and R₂, when taken together, constitute a substituted or unsubstituted 5- to 7-membered heterocyclic ring including the guanidinium group to which they are attached,

R₄ represents a substituted or unsubstituted 6- to 10-membered aromatic and heteroaromatic ring, and

X⁻ is an anion.

The uronium salt presents chemical formula (b):

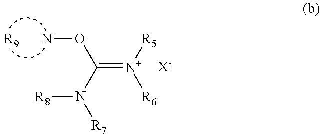

wherein

R₅, R₆, R₇, R₈ are independently selected from hydrogen, substituted or unsubstituted alkyl having 1 to 6 carbon atoms, substituted or unsubstituted alkenyl having 2 to 6 carbon atoms and substituted or unsubstituted alkenyl having 2 to 6 carbon atoms, or

R₅ and R₆, taken together, or R₇ and R₈, when taken together, constitute a substituted or unsubstituted 5- to 7-membered heterocyclic ring including the nitrogen atom to which they are attached, or R₆ and R₇, when taken together, constitute a substituted or unsubstituted 5- to 7-membered heterocyclic ring including the uronium group to which they are attached,

R₉ represents a substituted or unsubstituted 5- to 7-membered heterocyclic ring including the nitrogen atom to which is attached,

X⁻ is an anion.

Advantageously, the guanidinium or uronium salt is chosen among the following, commercially available compounds: O-(benzotriazol-1-yl)-N,N,N′,N′-tetramethyl uronium hexafluorophosphate (HBTU), or O-(benzotriazol-1-yl)-N,N,N′,N′-tetramethyluronium tetrafluoroborate (TBTU), or O-(7-azabenzotriazol-l-yl)-N,N,N′,N′-tetramethyluronium hexafluorophosphate (HATU), or N,N,N′,N′-tetramethyl-O—(N-succinimidyl)uronium tetrafluoroborate (TSTU), or N,N,N′,N′-tetramethyl-O—(N-succinimidyl)uronium hexafluorophosphate (HSTU), (1-cyano-2-ethoxy-2-oxoethylidenaminooxy)dimethylamino-morpholin-carbenium exafluorophosphate (COMU). It is worth noting that the commonly used nomenclature for HATU, HBTU, TBTU compounds is misleading, since it attributes these compounds to the uronium structure, instead of the correct guanidinium structure of formula (a).

Reagent A and Reagent B are formulated either as solids or as solutions, and when they are formulated as solution the solvent is a polar aprotic, water miscible solvent selected from N,N-dimethylformamide, dimethylsulfoxide, N,N-dimethylacetamide, N-methylpyrrolidone, acetonitrile; at least one of the two reagents is formulated as a solution.

The amount of tertiary amine used in the formulation of reagent A is from 1:1 to 10:1 molar ratio with respect to the carboxylated labelling compound, while the amount of coupling reagent used in the formulation of reagent B is from 1:1 to 10:1 molar ratio with respect to the carboxylated labelling compound.

The kit components can be stored until needed for a prolonged period. Advantageously, the kit is proportioned for a specific amount of the biomolecule to be labelled, so that weighing or aliquoting is not required, and only the necessary amount of labelling compound is used.

Activation of the carboxylated labelling compound occurs upon contacting Reagent A and Reagent B. The activation is carried out at a temperature between 0 and 60° C. for up to 60 minutes.

An especially advantageous application of the labelling kit of the present invention is in the labelling of proteins for 2-D Gel Electrophoresis; see, e.g. “Proteomics” by Renders and Sickman, Eds., Methods in Molecular Biology, Vol. 564, 2009, Humana Press. For this application, in fact, tiny amounts of active esters of three monocationic, size matched dyes in DMF solution are used commercially. In this form the have very limited stability and poor reproducibility, since one dye (pentamethinindocyanine) is more hydrolytically and photochemical unstable than the other two dyes (trimethinindocyanine and trimethinoxacyanine).

These problems are avoided by using the kit of this invention, where the three dyes are stored in a stable carboxylic acid form, which is activated only when needed.

Unless explicitly stated otherwise, the content of the references (articles, texts, patent applications, etc.) cited in this text is herein referred to in full for the sake of completeness of description. In particular, the mentioned references are herein incorporated by reference.

Further characteristics of the present invention will arise from the hereinafter description of some examples that are purely illustrative and not limiting.

### EXAMPLES

### Example 1

### Preparation of a Kit for the Labelling of Antibodies With a Fluorescent or Luminogenic Dye

The kit consists of two vials, one containing Reagent A, the other containing reagent B. The amount of reagents has been optimized for the labelling of 1 mg (6.7 nmol) of IgG (FR 150,000 daltons). Thus, the kit contains 15 equivalents of label (carboxylic acid) (100 nmol), 16.5 equivalents of 4-dimethylamino pyridine(DMAP) and 16.5 equivalents of N,N,′N′,-tetramethyl-O—(N-succinimidyl)uronium tetrafluoroborate (TSTU, 110 nmol) with respect to the IgG.

Reagent A (label+tertiary amine). Content: each 1.5 ml, conical bottom cryogenic vial with a o-ring seal cap contains 100 nmol of label (carboxylic acid) and 110 nmoles of DMAP.

Stock solutions are prepared by dissolving 2 μmoles of the label (carboxylic acid) in 1 ml of methanol or other volatile solvent and 1.07 mg (8.75 μmoles) of DMAP in 4 mL of methanol.

50 uL of each solution are added to a 1.5 mL conical bottom, cryogenic vial, and the solvent is evaporated using a rotary evaporator or an evaporative centrifuge.

The residue, which deposits as a film onto the bottom the vial, is further dried in vacuo.

The vial is then stored in the dark, under argon, at −20° C., in a desiccator.

Reagent B (activator). Each 0.5 mL, conical bottom cryogenic vial with a o-ring seal cap contains 70 μL of a 3 mM solution of TSTU in dry DMF.

A stock solution is prepared by dissolving 3.31 mg (11 μmoles) of TSTU in 5 mL of dry DMF under argon. 70 μL, aliquots are pipetted into 0.5 mL conical bottom, cryogenic vials, closed and stored under argon at −20° C., in a desiccator.

The kit is assembled by placing the vials containing Reagent A and Reagent B together with a desiccant bag inside a barrier-film (aluminium/polyethylene) pouch, which is then heat sealed and stored at -20° C.

### Example 2

### Labelling of an Antibody With a Fluorescent or Luminogenic Dye Using the Kit of Example 1

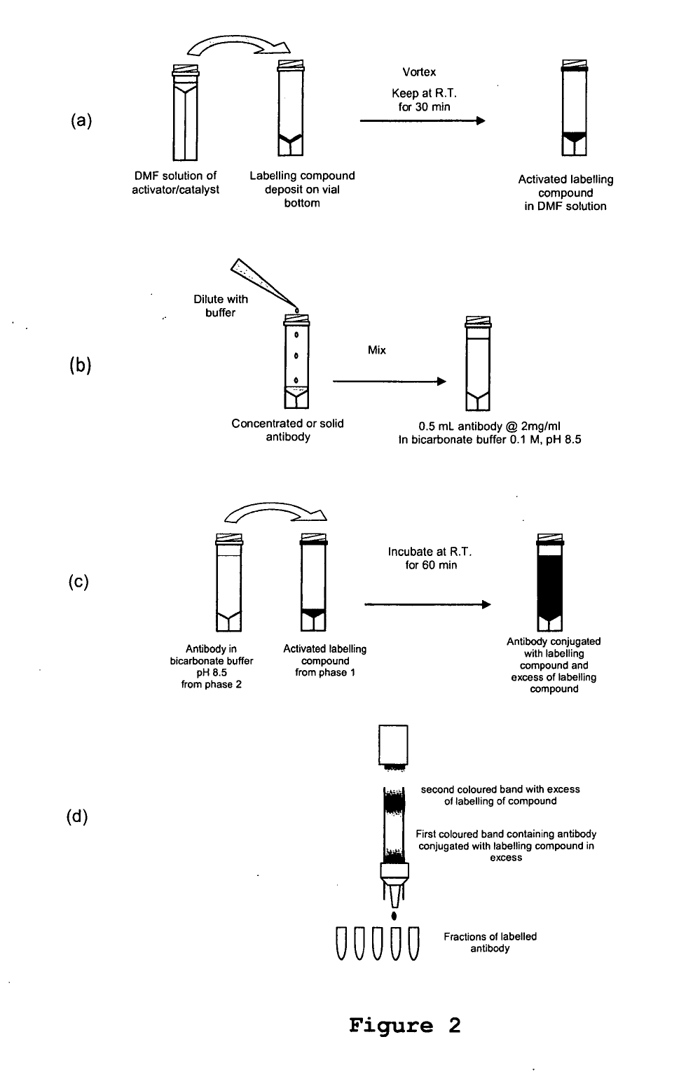

Step 1. Activated Dye Solution Preparation (FIG. 2a). the kit is warmed to room temperature. 50 μL of Reagent B (activator solution) are added to the vial containing Reagent A. The vial is capped, and the dye dissolved by vortexing; the mixture is kept in the dark at room temperature for 30 min (FIG. 2a).

Step 2. Antibody Solution Preparation (FIG. 2b). Each kit is designed to label 1 mg of IgG (MW 150,000) at 2 mg/mL, solution. The antibody must be dissolved in an amine-free buffer. For antibody buffer solution: to 450 uL, of antibody diluted to 2.2 mg/mL with PBS buffer, 50 uL of 1M, pH 8.5 bicarbonate buffer are added. For solid antibodies: 1 mg of antibody is dissolved in 0.5 mL of 0.1 M, pH 8.5 bicarbonate buffer (FIG. 2b).

Step 3. Conjugation Procedure (FIG. 2c). The antibody solution in buffer (from step 2) is added to the vial containing the dye solution (from step 1). The vial is capped, gently mixed (without vortexing) and the solution is incubated at room temperature in the dark for 1 h, shaking every 15 min.

Step 4. Purification and Isolation of the Conjugate (FIG. 2d). The conjugate product is separated from unreacted dye and reaction byproducts by gel permeation chromatography, e.g. on Sephadex™ G25. A chromatography column is equilibrated with PBS or the desired buffer, the reaction mixture from Step 3 is added, and eluted with the same buffer. The first colored band, containing the dye/antibody conjugate is collected. Alternatively one can use commercially available spin columns. Less efficient separation may be achieved by dialysis or ultrafiltration.

### Example 3

### Labelling of Proteins for 2-D Fluorescence Gel Electrophoresis (2-D DICE)

Labelling kits are specifically designed for proteins detection in 2-D Fluorescence Difference Gel Electrophoresis (2-D DICE) applications. The minimal labelling approach requires a minimized concentration of the dye in order to label each protein with a single molecule of dye. Labelling occurs by forming a covalent bond between the s-amino group of lysine in proteins and the activated dye.

For a typical minimal labelling 2D-DIGE analysis three dye labels are required, with approximate absorption wavelength at 490, 550 and 645 nm. The dyes must be:

monocationic. An intrinsic positive charge on each dye replaces the one from lysine leaving the total protein charge unchanged.

size-matched. All the dyes have similar molecular weight and shape to avoid affecting protein migration

pH insensitive: no signal affection is observed over a wide range of pH.

In addition, high brightness of the dyes is required, for high sensitivity.

For example, dyes belonging to the class of cyanines can fulfil this requirement.

Kit Preparation

Three separate kits are prepared, one for each dye. Each kit consists of two vials. One vial contains the dye/tertiary amine mixture, while the other contains the activator. For example, kits designed for 20 nmol of dye can be prepared as follows:

2-D DICE Dye/tertiary amine Vial. Three DIGE dyes are used, a monocationic pentamethinindocyanine, a monocationic trimethinindocyanine and a monocationic trimethinoxacyanine, respectively. The following stock solutions are prepared: 1.08 mg of DMAP are dissolved in 10 mL of dry DMF; 4 micromoles of DIGE dye are introduced into an amber 5-mL, volumetric flask, and 5 mL of the DMAP stock solution are added. The dye is dissolved, and the flask is capped under argon and stored at −20° C. 25 uL aliquots are pipetted into 0.5 mL, conical bottom, cryogenic vials. The vials are stored in the dark with a desiccant under argon at −20° C.

Activator Vial. Stock solution: 1.32 mg of TST are dissolved in 5 mL of anhydrous DM under argon in a 5 mL amber volumetric flask, in the dark with a desiccant under argon at −20° C. 25 uL aliquots are pipetted into 0.5 mL conical bottom, cryogenic vials. The vials are stored in the dark with a desiccant under argon at −20° C.

The kit is assembled by placing the two vials, together with a desiccant bag inside a barrier-film (aluminium/polyethylene) pouch, which is then heat sealed and stored at −20° C.

Each 2-DICE Minimal Labelling Kit contains 20 nmol of the 2-DICE Dye dissolved in 25 μL of free-amine dry DMF, allowing perform up to 50 labelling reactions.

The working solution is obtained by mixing equal volumes of the two supplied solutions. A typical labelling reaction on 50 μg of protein requires 1 μL of the labelling working solution, obtained by mixing 0.5 μL of 2-D DIGE Dye and 0.5 μL of Activator. To minimize pipetting errors the labelling reaction may be scaled-up as required.

Activation Procedure.

The kit is removed from the freezer and warmed up to room temperature for 5 minutes. The vials are inserted in a microcentrifuge and briefly spun to collect the solutions on the bottom of the vials. The labelling working solution is obtained as follows:

equal volumes of 2-D DIGS Dye/tertiary amine and activator solution are pipetted into a new microcentrifuge tube;

the tube is capped, mixed by vortexing and centrifuged for a few seconds, then kept the dark at room temperature for 30 minutes;

the labelling working solution will have a concentration of 400 pmol/μL in the active dye.

The working solution is typically prepared immediately before use. If necessary, the working solution can be stored at −20° C. for up to one week.

Labelling Protocol

The recommended protein concentration in the cell lysate is in the range 5-10 mg/mL, so that 50 μg of protein are portioned in 10 and 5 μL, respectively, but also samples containing 1 mg/mL of proteins may be labeled as well.

pH of the protein solution has to be 8.5 to guarantee the maximum efficiency.

To label 50 μg of protein 400 pmol of the active dye are recommended. Proceed as follow:

place the cell lysate solution containing 50 μg of protein in a microcentrifuge tube;

add 1 μL of the labelling working solution containing 400 pmol of the active dye to the same tube;

mix by pipetting up and down and centrifuge briefly;

incubate on ice for about 30 minutes in the darkness;

quench the labelling reaction by adding 1 μL of 10 mM lysine solution: mix by vortexing and centrifuge for few seconds, then keep in the dark on ice for 10 minutes.

Labelled protein solution is now ready to be processed, otherwise store at −70° C. in the darkness.

### Example 4

### Labelling of Amino-modified Oligonucleotides

A labelling kit was specifically developed to offer ideal conditions for the labelling of amino allyl modified DNA and RNA strands. The amount of label for each kit is adjusted to give an optimal 20-50 label molecules per 1000 nucleotides.

The kit consists of two vials, one containing Reagent A, the other containing reagent B. The amount of reagents has been optimized for the labelling of 5-20-mg (6.7 nmol) of aminoallyl arNA; aRNA=antisenseRNA. Thus, the kit contains 40 nmol of carboxylic acid label, 60 nmol of 4-dimethylamino-pyridine (DMAP) and 60 nmol of N,N,N′,N′-tetramethyl-O—(N-succinimidyl)uronium tetrafluoborate (TSTU) in dry DMF.

Reagent A (label+tertiary amine). Content: each 0.5 mL, DNase e RNase free, conical bottom, cryogenic vial with a o-ring seal cap contains 40 nmol of label (carboxylic acid) and 60 nmoles of DMAP. A stock solution is prepared by dissolving 7.2 μmoles of carboxylic acid label and 1.31 mg of DMAP in 5 mL of methanol in a 5 mL amber vial. 27.8 μL aliquots of this solution are pipetted into a 0.5 mL, conical bottom, cryogenic vial, and the solvent is evaporated using a rotary evaporator or an evaporative centrifuge. The residue, which deposits as a film onto the bottom the vial, is further dried in vacuo. The vial is then stored in the dark, under argon, at −20° C., in a dessicator.

Reagent B (activator). Content: each 0.5 mL, DNase e RNase free, conical bottom, cryogenic vial with a o-ring seal cap contains 20μL a TSTU solution. A stock solution is prepared by dissolving 8.20 mg of TSTU in 5 mL of dry DMF, under argon, in a 5 ml glass vial. 20.0 μL aliquots of this solution are pipetted into a 0.5 mL, conical bottom, cryogenic vial. The vial is then stored in the dark, under argon, at −20° C., in a dessicator.

The kit is assembled by placing the vials containing Reagent A and Reagent B together with a dessicant bag inside a barrier-film (aluminium(/polyethylene) pouch, which is then heat sealed and stored at −20° C.

Labelling protocol—The following protocol was developed for labelling amino allyl modified nucleotides using the labelling kit.

Step 1—Activated Label Solution Preparation. Let the kit warm to room temperature. Add 11 μL, of Reagent S (activator) solution to the Reagent A (label+tertiary amine) vial. Cap the vial, dissolve the dye by vortexing and keep in the dark at room temperature for 30 minutes.

Step 2—aRNA Solution Preparation. Each kit is designed to label room temperature. Each kit is designed to label 5-20 μg of amino allyl modified aRNA. Dry the desired amount of aRNA sample, using for example a vacuum centrifuge. Make sure not to overdry. Add 9 μL of coupling buffer (0.1 M sodium bicarbonate solution pH 8.7).

Step 3 Conjugation Procedure. Add the entire activated CHROMIS dye (11 μL from step 1) to the aRNA tube to bring the total volume to 20 μL. Pipette several times to mix well and incubate in the dark at room temperature for at least 30-60 minutes. To quench the reaction add 4.5 μL of a 4 M hydroxylamine solution and incubate in the dark at room temperature for 15 minutes. Proceed to purification of labelled aRNA probes by HPLC or using suitable purification cartridges and protocols.

Of course, without prejudice to the underlying principle of the invention, the details of construction and the embodiments may vary, even to a great extent, with respect to what has been described and illustrated purely by way of example, without departing from the scope of the present invention as defined by the annexed claims.

1.-18. (canceled)

19. A method for labelling biomolecules bearing reactive amino or hydroxyl groups with a carboxylated labelling compound, comprising
i) providing a mixture of the carboxylated labelling compound and an amine, and one between:
a guanidinium salt of formula (a) 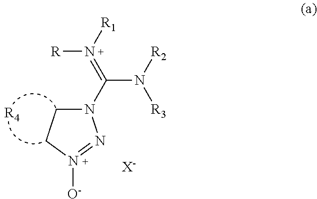
wherein
R, R₁, R₂, R₃ are independently selected from hydrogen, substituted or unsubstituted alkyl having 1 to 6 carbon atoms, substituted or unsubstituted alkenyl having 2 to 6 carbon atoms and substituted or unsubstituted alkynyl having 2 to 6 carbon atoms, or
R and R₁, when taken together, or R₂and R₃, when taken together, constitute a substituted or unsubstituted 5- to 7-membered heterocyclic ring including the nitrogen atom to which they are attached, or
R and R₂, when taken together, constitute a substituted or unsubstituted 5- to 7-membered heterocyclic ring including the guanidinium group to which they are attached, and
R₄ represents a substituted or unsubstituted 6- to 10-membered aromatic and heteroaromatic ring, and
X⁻ is an anion;
or
a uronium salt of formula (b) 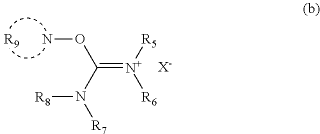
wherein
R₅, R₆, R₇, R₈are independently selected from hydrogen, substituted or unsubstituted alkyl having 1 to 6 carbon atoms, substituted or unsubstituted alkenyl having 2 to 6 carbon atoms and substituted or unsubstituted alkynyl having 2 to 6 carbon atoms, or
R₅and R₆, taken together, or R₇and R₈, when taken together, constitute a substituted or unsubstituted 5- to 7-membered heterocyclic ring including the nitrogen atom to which they are attached, or R₆ and R₇, when taken together, constitute a substituted or unsubstituted 5- to 7-membered heterocyclic ring including the uronium group to which they are attached, and
R₉ represents a substituted or unsubstituted 5- to 7-membered heterocyclic ring including the nitrogen atom to which is attached, and
X⁻ is an anion;
ii) contacting the mixture and the guanidinium salt or the uronium salt obtaining an activated carboxylated labelling compound, wherein the activated carboxylated labelling compound is not subjected to any intermediate purification;
iii) contacting the activated carboxylated labelling compound with the biomolecule obtaining a biomolecule labelled with the carboxylated labelling compound.

20. The method according to claim 19, wherein the operation ii) is performed at a temperature between 0 and 60° C. per a period of time up to 60 minutes.

21. The method according to claim 19, wherein the operation iii) is performed at a temperature between 4 and 50° C. per a period of time up to 24 hours.

22. The method according to claim 19, wherein the carboxylated labelling compound is a luminescent compound containing a carboxylic moiety.

23. The method according to claim 22, wherein the luminescent compound is selected from coumarines containing a carboxylic moiety, fluoresceines containing a carboxylic moiety and rhodamines containing a carboxylic moiety, polymethines containing a carboxylic moiety, diazaindacenes, amino substituted 2,3-dihydrophthalazine-1,4-diones, amino substituted 2,3-dihydrobenzo[f]phthalazine-1,4-diones, acridinium esters containing a carboxylic moiety, luminescent complexes of Ru (II), Os (II) and Ir (III) with aromatic diazines containing a carboxylic moiety, luminescent complexes of Eu (III) and Tb (III) containing a carboxylic moiety, a carboxyl functionalized derivative of luminol.

24. The method according to claim 19, wherein the amine is selected from triethylamine, N,N-diisopropylethylamine, pyridine, N,N-dimethylamino pyridine, 2,4,6-trimethylpyridine(collidine), 4-methyl morpholine, 4-ethyl morpholine, 4-morpholinopyridine.

25. The method according to claim 19, wherein the guanidinium salt or the uronium salt is used in 1:1 to 10:1 molar ratio with respect to said carboxylated labelling compound.

26. The method according to claim 19, wherein the tertiary amine is used in 1:1 to 10:1 molar ratio with respect to the carboxylated labelling compound.

27. The method according to claim 19, wherein said ganidinium salt of formula (a) is selected from O-(benzotriazol-1-yl)-N,N,N′,N′-tetramethyluronium hexafluorophosphate (H BTU), O-(benzotriazol-1-yl)-N,N,N′,N′-tetramethyl uroniu m tetrafluoroborate (TBTU), O-(7-azabenzotriazol-1-yl)-N,N,N′,N′-tetramethyluronium hexafluorophosphate (HATU).

28. The method according to claim 19, wherein said uronium salt of formula (b) is selected from N,N,N′,N′-tetra methyl-O—(N-succinimidyl)uronium tetrafluoroborate (TSTU), or N,N,N′,N′-tetra methyl-O—(N-succinimidyl)uronium hexafluorophosphate (HSTU), (1-cyano-2-ethoxy-2-oxoethylidenaminooxy)dimethylamino-morpholin-carbenium exafluorophosphate (COMU).

29. The method according to claim 23, wherein the polymethines comprise cyanines, merocyanines, oxazines, thiazines, or phthalo-and naphtha locyanines.

30. A method for labelling biomolecules bearing reactive amino or hydroxyl groups with a carboxylated labelling compound, comprising
i) providing a mixture of the carboxylated labelling compound and an amine, and one between:
a guanidinium salt selected from O-(benzotriazol-1-yl)-N,N′,N′,-tetramethyl uronium hexafluorophosphate (HBTU), O-(benzotriazol-1-yl)-N,N′,N′,W-tetramethyluronium tetrafluoroborate (TBTU), O-(7-azabenzotriazol-1-yl)-N,N,N′,N′-tetramethyluronium hexafluorophosphate (HATU);
or
an uronium salt selected from N,N,N′,N′,W-tetramethyl-O—(N-succinimidy)puronium tetrafluoroborate (TSTU), or N,N,N′,N′-tetramethyl-O—(N-succinimidyl)uronium hexafluorophosphate (HSTU), (1-cyano-2-ethoxy-2-oxoethylidenaminooxy)dimethylamino-morpholin-carbenium hexafluorophosphate (COMU),
ii) contacting the mixture and the guanidinium salt or the uronium salt obtaining an activated carboxylated labelling compound, wherein the activated carboxylated labelling compound is not subjected to any intermediate purification;
iii) contacting the activated carboxylated labelling compound with the biomolecule obtaining a biomolecule labelled with the carboxylated labelling compound;
wherein the amine is used in 1:1 to 10:1 molar ratio with respect to the carboxylated labelling compound,
wherein the guanidinium salt or the uronium salt is used in 1:1 to 10:1 molar ratio with respect to the carboxylated labelling compound,
wherein the carboxylated labelling compound is a luminescent compound.

31. The method according to claim 30, wherein the operation ii) is performed at a temperature between 0 and 60° C. per a period of time up to 60 minutes.

32. The method according to claim 30, wherein the operation iii) is performed at a temperature between 4 and 50° C. per a period of time up to 24 hours.

33. The method according to claim 30, wherein the luminescent compound is selected from coumarines containing a carboxylic moiety, fluoresceines containing a carboxylic moiety and rhodamines containing a carboxylic moiety, polymethines containing a carboxylic moiety, diazaindacenes, amino substituted 2,3-dihydrophthalazine-1,4-diones, amino substituted 2,3-dihydrobenzo[f]phthalazine-1,4-diones, acridinium esters containing a carboxylic moiety, luminescent complexes of Ru (II), Os (II) and Ir (III) with aromatic diazines containing a carboxylic moiety, luminescent complexes of Eu (III) and Tb (III) containing a carboxylic moiety, a carboxyl functionalized derivative of luminol.

34. The method according to claim 30, wherein the amine is selected from triethylamine, N,N-diisopropylethylamine, pyridine, N,N-dimethylamino pyridine, 2,4,6-trimethylpyridine(collidine), 4-methyl morpholine, 4-ethylmorpholine, 4-morpholinopyridine

35. A method for labelling biomolecules bearing reactive amino or hydroxyl groups with a carboxylated labelling compound, comprising
i) providing a mixture of the carboxylated labelling compound and an amine, an uronium salt selected from N,N,N′,N′-tetramethyl-O—(N-succinimidyl)uronium tetrafluoroborate (TSTU), or N,N,N′,N′-tetramethyl-O—(N-succinimidyl)uronium hexafluorophosphate (HSTU), (1-cyano-2-ethoxy-2-oxoethylidenaminooxy)dimethylamino-morpholin-carbenium hexafluorophosphate (COMU),
ii) contacting the mixture and the guanidinium salt or the uronium salt obtaining an activated carboxylated labelling compound, wherein the activated carboxylated labelling compound is not subjected to any intermediate purification;
iii) contacting the activated carboxylated labelling compound with the biomolecule obtaining a biomolecule labelled with the carboxylated labelling compound;
wherein the amine is used in 1:1 to 10:1 molar ratio with respect to said carboxylated labelling compound,
wherein the uronium salt is used in 1:1 to 10:1 molar ratio with respect to said carboxylated labelling compound,
wherein said carboxylated labelling compound is a luminescent compound,
wherein the luminescent compound is selected from coumarines containing a carboxylic moiety, fluoresceines containing a carboxylic moiety and rhodamines containing a carboxylic moiety, polymethines containing a carboxylic moiety, diazaindacenes, amino substituted 2,3-dihydrophthalazine-1,4-diones, amino substituted 2,3-dihydrobenzo[f]phthalazine-1,4-diones, acridinium esters containing a carboxylic moiety, luminescent complexes of Ru (II), Os (II) and Ir (III) with aromatic diazines containing a carboxylic moiety, luminescent complexes of Eu (III) and Tb (III) containing a carboxylic moiety, a carboxyl functionalized derivative of luminol, and
wherein the amine is selected from triethylamine, N,N-diisopropylethylamine, pyridine, N,N-di methyla mi no pyridine, 2,4,6-trimethylpyridine(collidine), 4-methyl morpholine, 4-ethyl morpholine, 4-morpholinopyridine.

36. The method according to claim 35, wherein the polymethines comprise cyanines, merocyanines, oxazines, thiazines, or phthalo-and naphthalocyanines.

37. The method according to claim 35, wherein the operation ii) is performed at a temperature between 0 and 60° C. per a period of time up to 60 minutes.

38. The method according to claim 35, wherein the operation iii) is performed at a temperature between 4 and 50° C. per a period of time up to 24 hours.
